(19) United States
(12) Patent Application Publication
(10) Pub. No.: US 2013/0110038 A1
(43) Pub. Date: May 2, 2013

(54) INFUSION APPARATUS AND COUNTERCURRENT PUSHBACK INFUSION APPARATUS

(71) Applicant: Chia-Yu Hsu, Taichung City (TW)

(72) Inventor: Chia-Yu Hsu, Taichung City (TW)

(21) Appl. No.: 13/286,189

(22) Filed: Oct. 31, 2011

(51) Int. Cl. A61M 5/168

(52) U.S. Cl. 604/83; 604/132

(57) ABSTRACT

A countercurrent pushback infusion apparatus includes an infusion pipe, a bag-shaped elastic container, a first blocker, and a second blocker. The infusion pipe has an output end, an input end, and a flow channel interconnecting the output and input ends. The bag-shaped elastic container is set on the infusion pipe and has an interior chamber connected to the flow channel. The first blocker is set on the infusion pipe between the bag-shaped elastic container and the output end and is switchable between a released position and a closed position. The second blocker is set on the infusion pipe between the bag-shaped elastic container and the input end and is switchable between a released position and a closed position. The apparatus allows pushback of blood countercurrent without the detachment of the needle and the infusion pipe, reduces the risk of infection, and increases the convenience of use.

### BACKGROUND

1. Technical Field

The invention relates generally to medical equipment, and more particularly, to an infusion apparatus and a countercurrent pushback infusion apparatus.

2. Related Art

When a medical worker is injecting infusion liquid, such as physiologic saline, medicine, or nutrient, into a patient's vein, the medical worker can used a syringe that is pressed manually or by a pump. Alternatively, the medical worker can hang an infusion bag above the patient's body, allowing gravity to force the infusion liquid into the patient's body through an infusion pipe and a needle.

However, in the infusion process, if the liquid drops too slowly, or the vein's pressure is too large, or the infusion bag's position is too low, some blood may reversely flow into the infusion pipe. If this happens, the medical worker needs to detach the needle and the infusion pipe, pour out the blood from the infusion pipe, use another syringe to draw the infusion liquid out from the infusion bag, detach the needle of this additional syringe, connect the syringe with the needle that is already in the patient's body, and use the infusion liquid to press the blood (that has flown into the needle) back into the vein.

The process mentioned above is not only time consuming but also will affect the medical worker's other duties. Furthermore, the process may cause either the patient, or the medical worker, or both, to be infected. Moreover, the process requires additional syringe and needle that will be disposed immediately after usage. Hence the process will not only waste medical resources but also increase medical costs.

Some inventions have been proposed to resolve the aforementioned problems. For example, the invention disclosed in a China Patent Application, the number of which is 200520080527.6 (CN2812952Y), proposes a manual quick-infusion connection pipe that can prevent blood countercurrent during infusion. The connection pipe has an elastic compression ball in its middle part. An anti-countercurrent pipe on a liquid inlet pipe is located inside the compression ball. An anti-countercurrent pipe on a liquid outlet pipe is located inside a ball funnel. The compression ball is connected to the liquid inlet pipe and the liquid outlet pipe. When the apparatus is infusing liquid normally, the liquid infusion can be expedited whenever necessary. After liquid infusion the apparatus can prevent blood countercurrent from blocking the puncturing needle.

When infusing liquid, the drip must have some minimum speed. Otherwise, there will be blood countercurrent if the vein's pressure is higher than the infusion liquid's pressure. Although the invention mentioned above uses two anti-countercurrent pipes to prevent blood countercurrent, the pipes also lower down the speed and flow of the infusion liquid, making it easier for blood to flow into the connection pipe. The reduced speed and flow of infusion also affect the supply of infusion liquid. If the infusion liquid contains prescription, such as medicine or anaesthetic, the reduced speed and flow of the infusion liquid will further affect the patient's therapy.

### BRIEF SUMMARY

Embodiments of the invention provide an infusion apparatus and a countercurrent pushback infusion apparatus. The apparatuses can push blood countercurrent back into a human body. To perform the pushback procedure, there is no need to detach a needle and an infusion pipe; hence it's less likely that a patent or a medical worker will be infected.

Embodiments of the invention further provide an infusion apparatus and a countercurrent pushback infusion apparatus, making a procedure of pushing back blood countercurrent relatively simpler and less time-consuming.

Embodiments of the invention further provide an infusion apparatus and a countercurrent pushback infusion apparatus, allowing a procedure of pushing back blood countercurrent to be performed without using additional medical tools. Hence the apparatuses can prevent medical resources from being wasted and medical costs from increasing.

Embodiments of the invention further provide an infusion apparatus and a countercurrent pushback infusion apparatus, allowing a function of pushing back blood countercurrent to not obstruct the flow and speed of infusion liquid flowing into a human body.

Embodiments of the invention further provide a countercurrent pushback infusion apparatus. The apparatus includes an infusion pipe, a bag-shaped elastic container, a first blocker, and a second blocker. The infusion pipe has an output end, an input end, and a flow channel interconnecting the output end and the input end. The bag-shaped elastic container is set on the infusion pipe. Inside the bag-shaped elastic container there is a chamber connected to the flow channel. The first blocker is set on a position of the infusion pipe between the bag-shaped elastic container and the output end and is switchable between a released position and a closed position. The second blocker is set on a position of the infusion pipe between the bag-shaped elastic container and the input end and is switchable between a released position and a closed position.

According to the invention, the output end of the infusion pipe is connected to a needle, the input end is connected to an infusion bag, and the input end and the output end allow infusion liquid in the infusion bag to flow through the flow channel of the infusion pipe and the needle and enter a human body.

The bag-shaped elastic container is integrated with the infusion pipe on a third position of the infusion pipe between the output end and the input end.

The bag-shaped elastic container has elasticity that allows the bag-shaped elastic container to be deformed and then restore its original shape automatically.

The first blocker and the second blocker are selected from switches, clamping components, and valves.

When the first blocker is at the first released position, the flow channel between the output end of the infusion pipe and the bag-shaped elastic container is unobstructed, and when the first blocker is at the first closed position, the flow channel between the output end of the infusion pipe and the bag-shaped elastic container is obstructed.

When the second blocker is at the second released position, the flow channel between the input end of the infusion pipe and the bag-shaped elastic container is unobstructed, and when the second blocker is at the second closed position, the flow channel between the input end of the infusion pipe and the bag-shaped elastic container is obstructed.

The bag-shaped elastic container comprises a medicine adder.

The medicine adder and the bag-shaped elastic container form an integrated piece, material of the medicine adder and the bag-shaped elastic container is selected from rubber, silica gel, and elastoplastic.

The material of the bag-shaped elastic container is selected from rubber, silica gel, and elastoplastic.

When there is blood countercurrent, the second blocker is switched to the second closed position, then the bag-shaped elastic container is pressed to push the blood countercurrent back to a human body, next the first blocker is switched to the first closed position to prevent further countercurrent; thereafter, the second blocker is switched to the second released position, the restoring force of the bag-shaped elastic container sucks into the infusion liquid from the input end of the infusion pipe, then the first blocker is switched to the first released position, making the flow channel of the infusion pipe to become unobstructed.

On the other hand, embodiments of the invention further provide an infusion apparatus that incorporates the aforementioned countercurrent pushback infusion apparatus.

The invention can prevent blockage caused by blood countercurrent, avoid repetitive punctures of needles, and alleviate the patients' pain. The bag-shaped elastic container can be used to push blood countercurrent back into the human body, reduce the number of required syringes and needles, and as a result preserve medical resources and costs. Furthermore, the invention does not reduce the flow or speed of the infusion liquid and hence will not obstruct infusion liquid from flowing into the human body. As a result, the invention does not cause problems related to insufficient supply of infusion liquid. When being used, there is no need to detach the needle and the infusion pipe; hence the invention can reduce the patient and medical worker's risks of infection. The apparatuses of the invention are not only easy to use, but can also save time, increase convenience, and reduce medical workers' work load.

Other features of the present invention will be apparent from the accompanying drawings and from the detailed description which follows.

### BRIEF DESCRIPTION OF THE DRAWINGS

The invention is fully illustrated by the subsequent detailed description and the accompanying drawings, in which like references indicate similar elements.

FIG. 1 shows a pictorial view of a detached apparatus according to a first embodiment of the invention.

FIG. 2 shows a pictorial view of an assembled apparatus according to the first embodiment of the invention.

FIG. 3 shows a cross sectional view of the assembled apparatus according to the first embodiment of the invention.

FIGS. 4-8 illustrate how to operate the apparatus according to the first embodiment of the invention.

FIG. 9 shows a pictorial view of an assembled apparatus according to a second embodiment of the invention.

FIG. 10 illustrates how to operate the apparatus according to the second embodiment of the invention.

### DETAILED DESCRIPTION

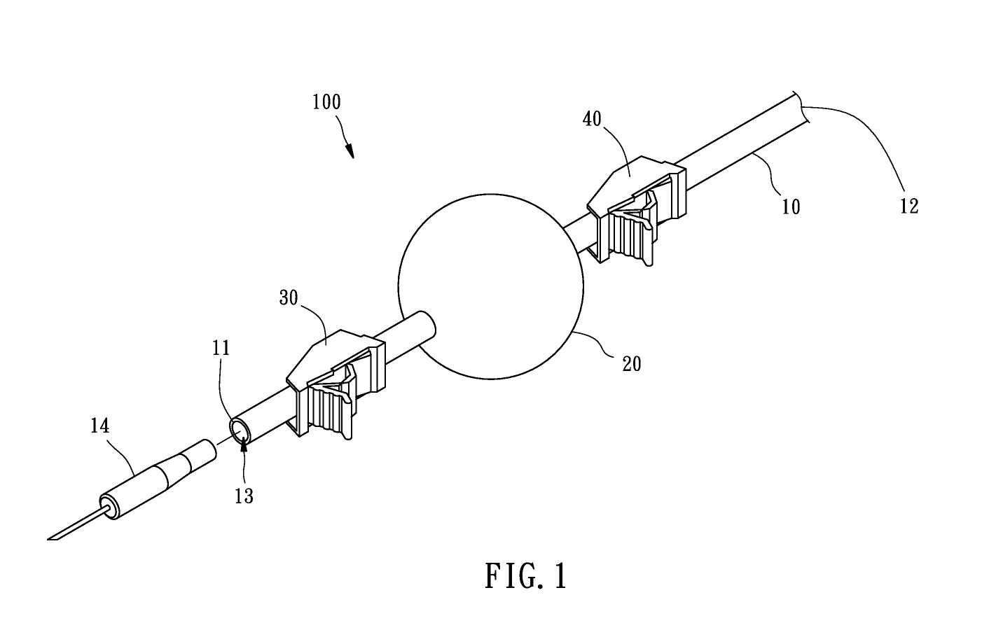

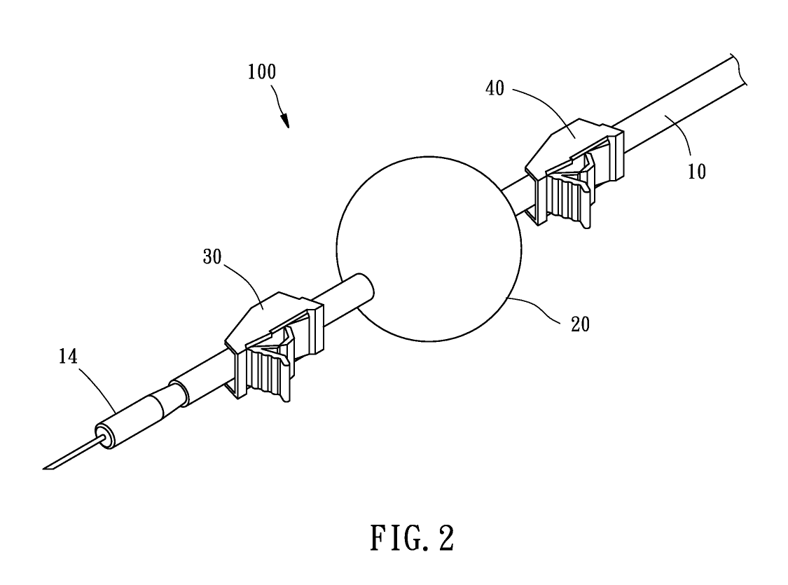

Please refer to FIGS. 1-8; they illustrate a countercurrent pushback infusion apparatus 100 according to an embodiment of the invention. The countercurrent pushback infusion apparatus 100 mainly includes an infusion pipe 10, a bag-shaped elastic container 20, a first blocker 30, and a second blocker 40.

Please refer to FIGS. 1-3. The infusion pipe 10 is a hollow pipe having an output end 11 and an input end 12. Inside the infusion pipe 10 there is a flow channel 13 that is connected to the output end 11 and the input end 12 and allows infusion liquid to flow through. The output end 11 of the infusion pipe 10 can be connected to a needle 14; the input end 12 can be connected to an infusion bag that is not depicted in the figures. The infusion liquid in the infusion bag can flow through the flow channel 13 of the infusion pipe 10 and the needle 14 and enter a vein of a human body.

Because the infusion bag can be a conventional one having conventional structure and functions, no further illustration will be provided for the infusion bag.

Please refer to FIGS. 1-3. The bag-shaped elastic container 20 is set on a position of the infusion pipe 10 between the output end 11 and the input end 12 and forms a single integrated piece together with the infusion pipe 10. The bag-shaped elastic container 20 can have elastic deformation and then resume its original shape using elastic force. Inside the bag-shaped elastic container 20 there is a chamber 21. Two sides of the chamber 21 are connected to the flow channel 13 of the infusion pipe 10. It's preferred that the bag-shaped elastic container 20 is close to the needle 14's position. The material of the bag-shaped elastic container 20 can be selected from rubber, silica gel, or other material with similar characteristics.

Please refer to FIGS. 1-3. The first blocker 30 is set on the infusion pipe 10 and lies between the bag-shaped elastic container 20 and the output end 11 of the infusion pipe 10. A medical worker can apply external force to the first blocker 30, causing it to switch from a closed position to a released position, or vice versa. In this embodiment, the first blocker 30 is a clamping component.

Please refer to FIGS. 1-3. The second blocker 40 is set on the infusion pipe 10 and lies between the bag-shaped elastic container 20 and the input end 12 of the infusion pipe 10. It's preferred that the second blocker 40 is close to the bag-shaped elastic container 20's position. A medical worker can apply external force to the second blocker 40, causing it to switch from a closed position to a released position, or vice versa. In this embodiment, the second blocker 40 is a clamping component.

The above paragraphs have illustrated the components of the countercurrent pushback infusion apparatus 100 and the assembly of the components. The following paragraphs will illustrate the characteristics of the countercurrent pushback infusion apparatus 100 when it's being use.

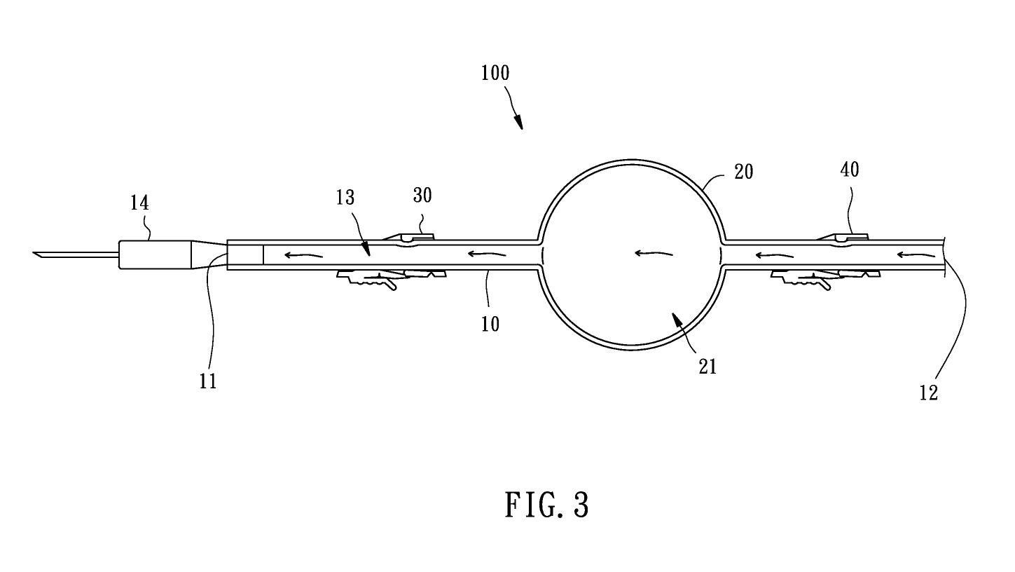

Under normal usage, the first blocker 30 and the second blocker 40 are in their released positions as depicted in FIG. 3. The infusion liquid inside the infusion bag can flow through the input end 12 of the infusion pipe 10, the flow channel 13, the output end 11, and the needle 14 in turn, and eventually reach the vein of a human body. Because the first blocker 30 and the second blocker 40 are in their released positions, they will not block the flow of the infusion liquid through the flow channel 13. As a result, the flow and speed of the infusion liquid will not be affected by the first blocker 30 and the second blocker 40.

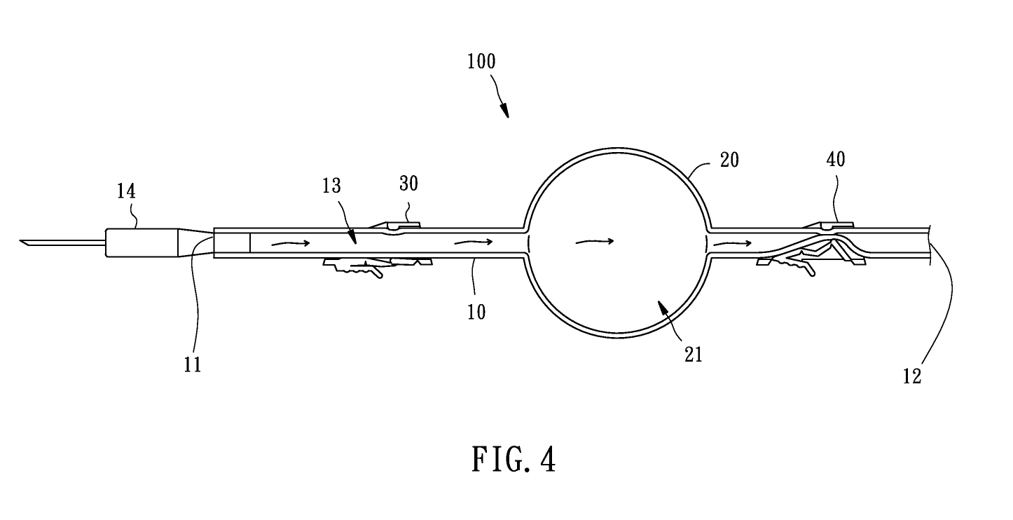

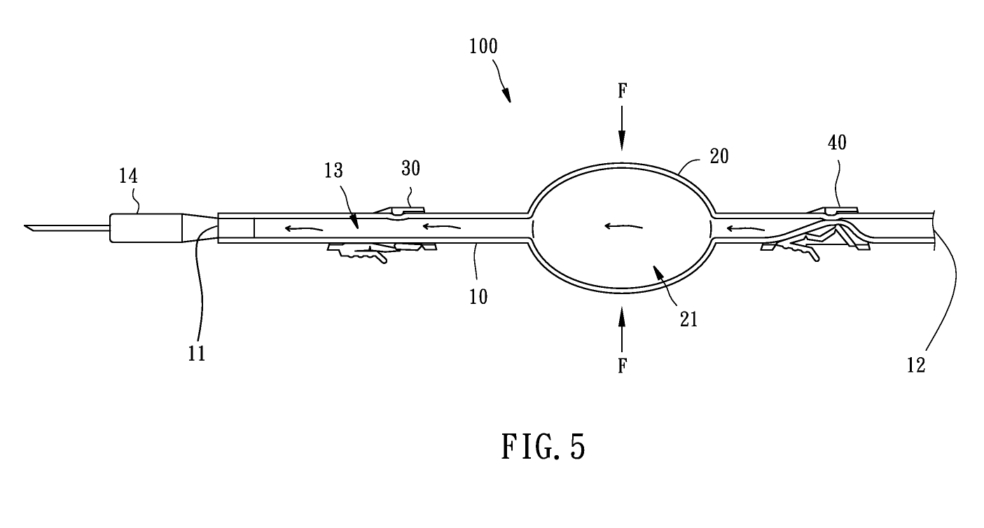

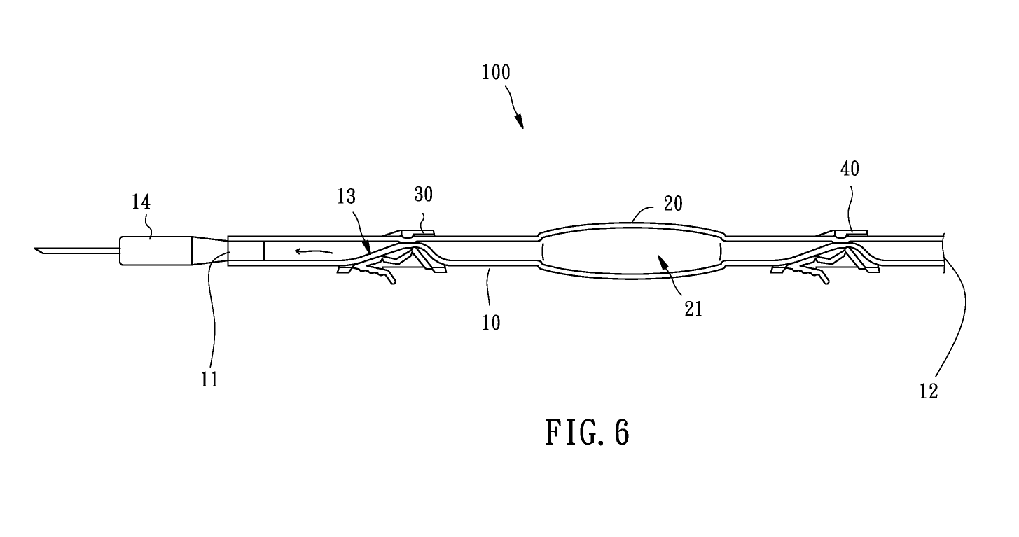

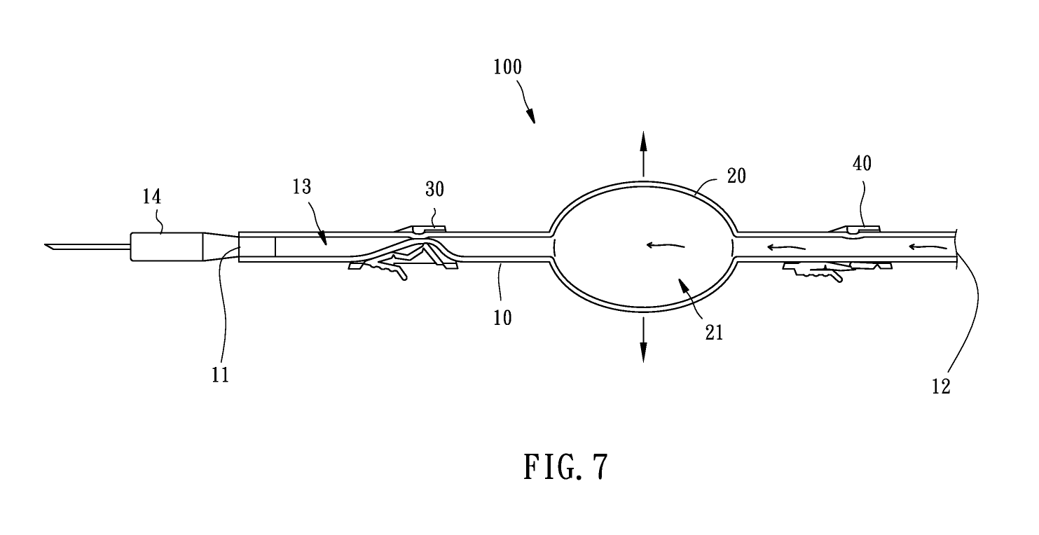

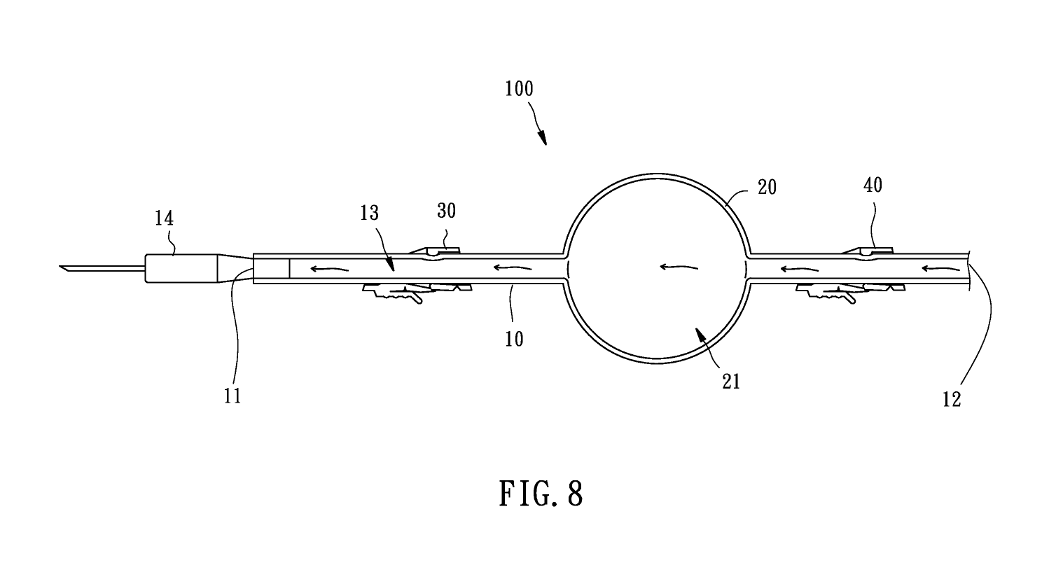

When there is blood countercurrent, i.e. some blood has flown from the human body into the infusion pipe 10 through the needle 14 and the output end 11, the medical worker using the countercurrent pushback infusion apparatus 100 first switches the second blocker 40 to its closed position, as shown in FIG. 4, preventing blood in the flow channel 13 from passing through the second blocker 40. Then, the medical worker can apply a force F to press the bag-shaped elastic container 20, as shown in FIG. 5, to increase the pressure inside the chamber 21 of the bag-shaped elastic container 20. The increased pressure will cause the infusion liquid and blood inside the chamber 21 and the part of the flow channel 13 between the second blocker 40 and the output end 11 to flow into the vein of the human body. Once the infusion liquid and blood inside the chamber 21 and the flow channel 13 between the second blocker 40 and the output end 11 has passed the first blocker 30, the medical worker can switch the first blocker 30 to its closed position, as shown in FIG. 6. Then, the first blocker 30 will prevent blood countercurrent from occurring again. Next, the medical worker can switch the second blocker 40 to its released position, allowing the restoring force of the bag-shaped elastic container 20 to suck the infusion liquid between the second blocker 40 and the infusion bag into the chamber 21, as shown in FIG. 7. Then, the medical worker can switch the first blocker 30 to its released position, allowing free flow inside the flow channel 13 of the infusion pipe 10, as shown in FIG. 8. Thereafter, the infusion liquid can normally flow from the infusion bag into the infusion pipe 10, and reach the human body through the needle 14.

The invention uses a bag-shaped elastic container to force blood countercurrent to flow back into a human body, and hence allows the problems and troubles caused by blood countercurrent to be resolved quickly. It further avoids blockage caused by the countercurrent. Not only is it convenient to push back countercurrent directly, but it also prevents the troublesome procedure of dealing with countercurrent. Furthermore, because the invention does not need addition needle, it can avoid wasting resources and can reduce medical costs. More importantly, if the push back components of the invention are not in use, they will not affect the flow channel of the infusion pipe. In other words, unlike some related arts, the infusion liquid will not be blocked but instead can maintain flow and speed, avoiding the related arts' problem of insufficient supply of infusion liquid. Moreover, the invention does not require a medical worker to detach a needle and an infusion pipe, hence protecting the patient and the medical worker from infection. Finally, the invention is easy to operate, can save its medical worker's time, increase the convenience of use, and reduce the medical worker's work load.

The first and second blockers are mainly used to open or close the flow channel of the infusion pipe. As a result, the first and second blockers can be embodied by any conventional components that can be opened or closed, such as switches, clamping components, and valves. These and other similar components can be used to realize the first and second blockers and achieve the objectives of the invention.

In combination, the aforementioned countercurrent pushback infusion apparatus 100 and the infusion bag form a great infusion apparatus.

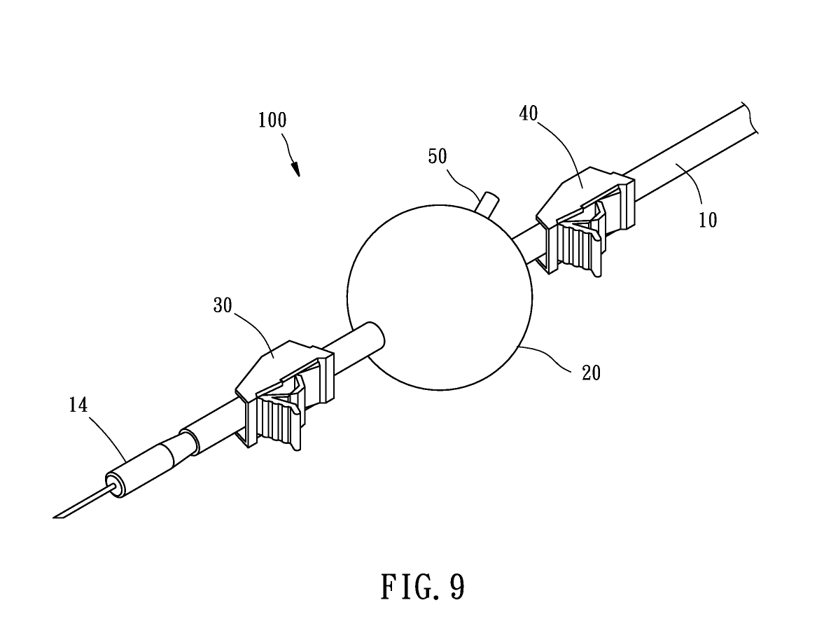

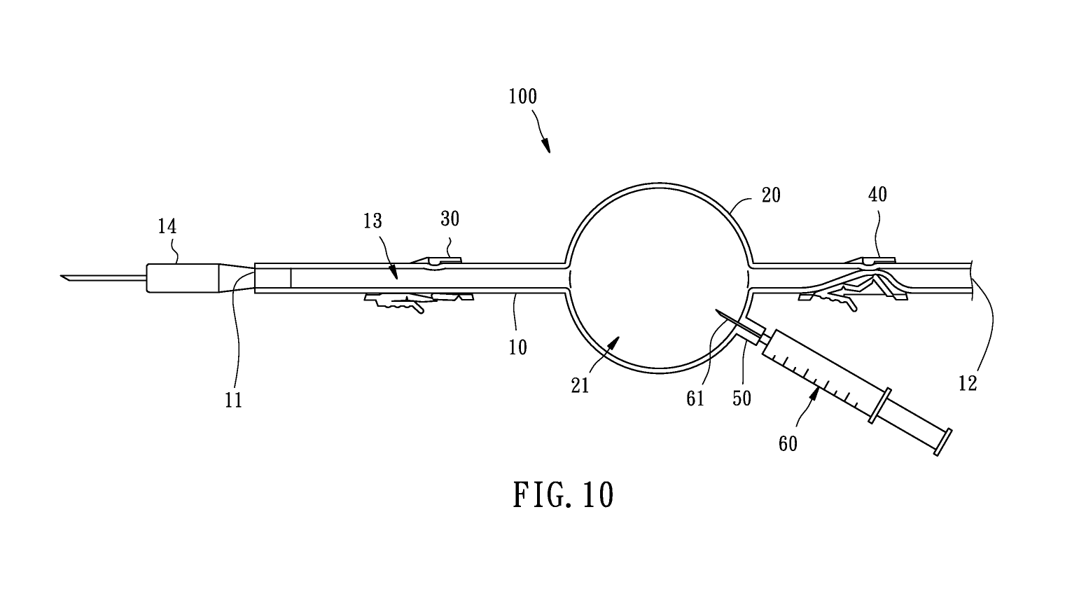

Please refer to FIGS. 9 and 10. They depict a pictorial view and a cross sectional view of a countercurrent pushback infusion apparatus according to another embodiment of the invention. The infusion apparatus 100 of this embodiment and that of the previous embodiment have some common components, and in FIGS. 9 and 10 these common components simply retain their numeral indexes used in the previous figures. One of the differences between these two embodiments is that in the second embodiment, the bag-shaped elastic container 20 further has a medicine adder 50. The medicine adder 50 allows medicine to be added into the infusion liquid. In this embodiment, the medicine adder 50 is an extruding part of the bag-shaped elastic container 20's main body. It and the bag-shaped elastic container 20 can be made up of the same material and can be integrated into a single pierce. Some exemplary materials of the medicine adder 50 include rubber, silica gel, elastoplastic, etc. To make it convenient for a needle to puncture through, stripe is a preferred shape of the medicine adder 50. Because the medicine adder 50 is made of elastic material, when the needle has been taken out, the hole caused by the needle will close automatically. To add medicine, a medical worker must first switch the second blocker 40 to its closed position, puncture the needle 61 of the medicine adding syringe 60 into the bag-shaped elastic container 20 through the medicine adder 50. Once the medicine in the medicine adding syringe 60 has been pressed into the bag-shaped elastic container 20, the needle 61 is taken out. Afterward, there are two alternative operations. In a first alternative, the medical worker presses the bag-shaped elastic container 20 to push the added medicine into the human body. In a second alternative, the medical worker can switch the second blocker 40 to its released position, allowing the added medicine to flow into the human body together with the infusion liquid. An advantage of these two alternatives is that they allow the medical worker to choose the speed the medicine flows into the human body. For some ordinary medicine, the medical worker can press the bag-shaped elastic container 20 directly, causing the medicine to enter the human body quickly. For some other medicine that may make the patient uncomfortable, such as heart medicine or antibiotic, the medical worker can allow the medicine to flow into the human body slowly together with the infusion liquid, making the patient less uncomfortable.

In the foregoing detailed description, the invention has been described with reference to specific exemplary embodiments thereof. It will be evident that various modifications may be made thereto without departing from the spirit and scope of the invention as set forth in the following claims. The detailed description and drawings are, accordingly, to be regarded in an illustrative sense rather than a restrictive sense.

What is claimed is:

1. A countercurrent pushback infusion apparatus, comprising:
an infusion pipe, having an output end, an input end, and a flow channel interconnecting the output end and the input end;
a bag-shaped elastic container, set on the infusion pipe, having an interior chamber connected to the flow channel of the infusion pipe;
a first blocker, set on a first position of the infusion pipe between the bag-shaped elastic container and the output end, switchable between a first released position and a first closed position; and
a second blocker, set on a second position of the infusion pipe between the bag-shaped elastic container and the input end, switchable between a second released position and a second closed position.

2. The apparatus of claim 1, wherein the output end of the infusion pipe is connected to a needle, the input end is connected to an infusion bag, and the input end and the output end allow infusion liquid in the infusion bag to flow through the flow channel of the infusion pipe and the needle and enter a human body.

3. The apparatus of claim 1, wherein the bag-shaped elastic container is integrated with the infusion pipe on a third position of the infusion pipe between the output end and the input end.

4. The apparatus of claim 1, wherein the bag-shaped elastic container has elasticity that allows the bag-shaped elastic container to be deformed and then restore its original shape automatically.

5. The apparatus of claim 1, wherein the first blocker and the second blocker are selected from switches, clamping components, and valves.

6. The apparatus of claim 1, wherein when the first blocker is at the first released position, the flow channel between the output end of the infusion pipe and the bag-shaped elastic container is unobstructed, and when the first blocker is at the first closed position, the flow channel between the output end of the infusion pipe and the bag-shaped elastic container is obstructed.

7. The apparatus of claim 1, wherein when the second blocker is at the second released position, the flow channel between the input end of the infusion pipe and the bag-shaped elastic container is unobstructed, and when the second blocker is at the second closed position, the flow channel between the input end of the infusion pipe and the bag-shaped elastic container is obstructed.

8. The apparatus of claim 1, wherein the bag-shaped elastic container comprises a medicine adder.

9. The apparatus of claim 8, wherein the medicine adder and the bag-shaped elastic container form an integrated piece, material of the medicine adder and the bag-shaped elastic container is selected from rubber, silica gel, and elastoplastic.

10. The apparatus of claim 1, wherein material of the bag-shaped elastic container is selected from rubber, silica gel, and elastoplastic.

11. The apparatus of claim 1, wherein when there is blood countercurrent, the second blocker is switched to the second closed position, then the bag-shaped elastic container is pressed to push the blood countercurrent back to a human body, next the first blocker is switched to the first closed position to prevent further countercurrent; thereafter, the second blocker is switched to the second released position, the restoring force of the bag-shaped elastic container sucks into the infusion liquid from the input end of the infusion pipe, then the first blocker is switched to the first released position, making the flow channel of the infusion pipe to become unobstructed.

12. An infusion apparatus capable of pushing countercurrent back into a human body, comprising:
an infusion bag;
an infusion pipe, having an output end, an input end, and a flow channel interconnecting the output end and the input end, wherein the output end of the infusion pipe is connected to a needle, the input end is connected to an infusion bag, and the input end and the output end allow infusion liquid in the infusion bag to flow through the flow channel of the infusion pipe and the needle and enter the human body;
a bag-shaped elastic container, set on the infusion pipe, having an interior chamber connected to the flow channel of the infusion pipe;
a first blocker, set on a first position of the infusion pipe between the bag-shaped elastic container and the output end, switchable between a first released position and a first closed position; and
a second blocker, set on a second position of the infusion pipe between the bag-shaped elastic container and the input end, switchable between second a released position and a second closed position;
wherein when there is blood countercurrent, the second blocker is switched to the second closed position, then the bag-shaped elastic container is pressed to push the blood countercurrent back to the human body, next the first blocker is switched to the first closed position to prevent further countercurrent; thereafter, the second blocker is switched to the second released position, the restoring force of the bag-shaped elastic container sucks into the infusion liquid from the input end of the infusion pipe, then the first blocker is switched to the first released position, making the flow channel of the infusion pipe to become unobstructed.

13. The apparatus of claim 12, wherein when the first blocker is at the first released position, the flow channel between the output end of the infusion pipe and the bag-shaped elastic container is unobstructed, and when the first blocker is at the first closed position, the flow channel between the output end of the infusion pipe and the bag-shaped elastic container is obstructed.

14. The apparatus of claim 12, wherein when the second blocker is at the second released position, the flow channel between the input end of the infusion pipe and the bag-shaped elastic container is unobstructed, and when the second blocker is at the second closed position, the flow channel between the input end of the infusion pipe and the bag-shaped elastic container is obstructed.

15. The apparatus of claim 12, wherein the bag-shaped elastic container comprises a medicine adder.
